(19) United States
(12) Patent Application Publication
(10) Pub. No.: US 2005/0222054 A1
(43) Pub. Date: Oct. 6, 2005

(54) Combination comprising a CDK inhibitor and doxorubicin

(75) Inventors: Roger Neil Sleigh, Dundee (GB); Anton Berns, Amsterdam (NL); Helen Mary Coley, Guildford (GB); Scott Lyons, San Francisco, CA (US)

Correspondence Address: LAHIVE & COCKFIELD, LLP., 28 STATE STREET, BOSTON, MA, 02109, US

(73) Assignee: Cyclacel Limited, Dundee (GB)

(21) Appl. No.: 10/952,575

(22) Filed: Sep. 27, 2004

Related U.S. Application Data
(63) Continuation of application No. PCT/GB03/01282, filed on Mar. 25, 2003.

(30) Foreign Application Priority Data
Mar. 27, 2002 (GB) 0207228.8
Sep. 26, 2002 (GB) 0222408.7
Nov. 6, 2002 (GB) 0225876.2

(51) Int. Cl. A61K 31/704; A61K 31/522

(52) U.S. Cl. 514/34; 514/263.31

(57) ABSTRACT

A first aspect of the invention relates to a combination comprising a CDK inhibitor and doxorubicin. A second aspect of the invention relates to a pharmaceutical product comprising a CDK inhibitor and doxorubicin as a combined preparation for simultaneous, sequential or separate use in therapy. A third aspect of the invention relates to a method of treating a proliferative disorder, said method comprising simultaneously, sequentially or separately administering a CDK inhibitor and doxorubicin to a subject.

### FIELD OF THE INVENTION

The present invention relates to a pharmaceutical combination suitable for the treatment of cancer and other proliferative disorders.

### BACKGROUND TO INVENTION

Initiation, progression, and completion of the mammalian cell cycle are regulated by various cyclin-dependent kinase (CDK) complexes, which are critical for cell growth. These complexes comprise at least a catalytic (the CDK itself) subunit and a regulatory (cyclin) subunit. Some of the more important complexes for cell cycle regulation include cyclin A (CDK1—also known as cdc2, and CDK2), cyclin B1-B3 (CDK1), cyclin C (CDK8), cyclin D1-D3 (CDK2, CDK4, CDK5, CDK6), cyclin E (CDK2), cyclins K and T (CDK9) and cyclin H (CDK7). Each of these complexes is involved in a particular phase of the cell cycle.

The activity of CDKs is regulated post-translationally, by transitory associations with other proteins, and by alterations of their intracellular localisation. Tumour development is closely associated with genetic alteration and deregulation of CDKs and their regulators, suggesting that inhibitors of CDKs may be useful anti-cancer therapeutics. Indeed, early results suggest that transformed and normal cells differ in their requirement for e.g. cyclin A/CDK2 and that it may be possible to develop novel antineoplastic agents devoid of the general host toxicity observed with conventional cytotoxic and cytostatic drugs.

The function of CDKs is to phosphorylate and thus activate or deactivate certain proteins, including, for example, retinoblastoma proteins, lamins, histone H1, and components of the mitotic spindle. The catalytic step mediated by CDKs involves a phospho-transfer reaction from ATP to the macromolecular enzyme substrate. Several groups of compounds (reviewed in N. Gray, L. Détivaud, C. Doerig, L. Meijer, Curr. Med. Chem. 1999, 6, 859) have been found to possess anti-proliferative properties by virtue of CDK-specific ATP antagonism.

Roscovitine is the compound 6-benzylamino-2-[(R)-1-ethyl-2-hydroxyethylamino]-9-isopropylpurine. Roscovitine has been demonstrated to be a potent inhibitor of cyclin dependent kinase enzymes, particularly cdk2. This compound is currently in development as an anti-cancer agent. Cdk inhibitors are understood to block passage of cells from the G1/S and the G2/M phase of the cell cycle. Roscovitine has also been shown to be an inhibitor of retinoblastoma phosphorylation and therefore implicated as acting more potently on Rb positive tumors.

It well established in the art that active pharmaceutical agents can often be given in combination in order to optimise the treatment regime. The present invention therefore seeks to provide a new combination of known pharmaceutical agents that is particularly suitable for the treatment of proliferative disorders, especially cancer. More specifically, the invention centres on the surprising and unexpected effects associated with using certain pharmaceutical agents in combination.

### STATEMENT OF INVENTION

In a first aspect, the invention provides a combination comprising a CDK inhibitor and doxorubicin.

A second aspect provides a pharmaceutical composition comprising a combination according to the invention admixed with a pharmaceutically acceptable carrier, diluent or excipient.

A third aspect relates to the use of a combination according to the invention in the preparation of a medicament for treating a proliferative disorder.

A fourth aspect relates to a pharmaceutical product comprising a CDK inhibitor and doxorubicin as a combined preparation for simultaneous, sequential or separate use in therapy

A fifth aspect relates to a method of treating a proliferative disorder, said method comprising simultaneously, sequentially or separately administering a CDK inhibitor and doxorubicin to a subject.

A sixth aspect relates to the use of a CDK inhibitor in the preparation of a medicament for the treatment of a proliferative disorder, wherein said treatment comprises simultaneously, sequentially or separately administering a CDK inhibitor and doxorubicin to a subject.

A seventh aspect relates to the use of a CDK inhibitor and doxorubicin in the preparation of a medicament for treating a proliferative disorder.

An eighth aspect relates to the use of a CDK inhibitor in the preparation of a medicament for the treatment of a proliferative disorder, wherein said medicament is for use in combination therapy with doxorubicin.

A ninth aspect relates to the use of doxorubicin in the preparation of a medicament for the treatment of a proliferative disorder, wherein said medicament is for use in combination therapy with a CDK inhibitor.

### DETAILED DESCRIPTION

The preferred embodiments as set out below are applicable to all the above-mentioned aspects of the invention.

As mentioned above, the present invention relates to a combination comprising a CDK inhibitor and doxorubicin. Preferably, the combination is a synergistic combination. Doxorubicin is the compound (8S-cis)-8-acetyl-10-[(3-amino-2,3,6-trideoxy-alpha-L-lyxo-hexopyranosyl)oxy]7,8,9,10-tetrahydro-6,8,11-trihydroxy-1-methoxy-5,12-naphthacenedione which is an anthracycline antibiotic. Anthracycline antibiotics were first isolated from microorganisms in 1939, and their antibiotic properties were studied in the 1950s. These antibiotics killed bacteria quite readily, but were too toxic to be used for treating infections in humans. It was not until the 1960s that anthracycline antibiotics were tested for antitumor properties and found to be active against cancer cells.

The anthracyclines all bind to DNA and their cytotoxicity largely results from this binding. More specifically, they bind to double stranded DNA. In human chromosome preparations treated with anthracyclines the bound drug is observed as well-defined, orange-red fluorescent bands. This interaction with DNA is by intercalation and has been identified as such by several methods. If the structure of the anthracyclines is modified so as to alter the binding to DNA, there is usually a decrease or loss of antitumor activity. Thus, DNA binding seems to be critical for the anticancer activity of these drugs. However, the pathway leading to cytotoxicity has not been clearyly elucidated. Inhibition of DNA and RNA synthesis is not thought to be critical for cytotoxicity as it only occurs at high drug concentrations.

Anthracyclines have a number of important effects, any one or all of which have a role in the cytotoxic actions of these drugs. First of all, they can intercalate with DNA, thereby affecting many functions of the DNA, including DNA and RNA synthesis. Breakage of the DNA strand can also occur. This is believed to be mediated either by the enzyme DNA topoisomerase II or by the formation of free radicals. Inhibition of the enzyme topoisomerase II, for example, can lead to a series of reactions leading to double strand breaks in the DNA.

Temporary double-strand breaks are induced by topoisomerase II in the course of its normal catalytic cycle, by the formation of a cleavable complex. Disruption of this complex, which results in a permanent double-strand break, occurs infrequently in the absence of drugs. Inhibitors of topoisomerase II cause the cleavable complex to persist, thereby increasing the probability that the cleavable complex will be converted to an irreversible double-strand break.

Anthracyclines can also cause the formation of active oxygen species that then cause predominantly single-strand breakage. The anthracycline chromophore contains a hydroxyquinone, which is a well-described iron chelating structure. The drug-Fe-DNA complex catalyzes the transfer of electrons from glutathione to oxygen, resulting in the formation of active oxygen species.

Another aspect of the invention relates to a combination comprising a CDK inhibitor and an anthracycline.

Yet another aspect relates to a pharmaceutical product comprising a CDK inhibitor and an anthracycline.

As mentioned above, one aspect of the invention relates to a pharmaceutical product comprising a CDK inhibitor and doxorubicin as a combined preparation for simultaneous, sequential or separate use in therapy.

The CDK inhibitor and doxorubicin may be administered simultaneously, in combination, sequentially or separately (as part of a dosing regime).

As used herein, “simultaneously” is used to mean that the two agents are administered concurrently, whereas the term “in combination” is used to mean they are administered, if not simultaneously, then “sequentially” within a timeframe that they both are available to act therapeutically within the same time-frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction therebetween, and their respective half-lives.

In contrast to “in combination” or “sequentially”, “separately” is used herein to mean that the gap between administering one agent and the other is significant i.e. the first administered agent may no longer be present in the bloodstream in a therapeutically effective amount when the second agent is administered.

Preferably, the CDK inhibitor and doxorubicin are administered simultaneously or sequentially.

One aspect of the present invention relates to the use of a CDK inhibitor in the preparation of a medicament for the treatment of a proliferative disorder, wherein said treatment comprises administering to a subject simultaneously, sequentially or separately doxorubicin and a CDK inhibitor.

In one particularly preferred embodiment, the CDK inhibitor is administered to the subject prior to sequentially or separately administering doxorubicin to said subject.

In an alternative preferred embodiment, doxorubicin is administered to the subject prior to sequentially or separately administering a CDK inhibitor to said subject.

In one preferred embodiment of the invention, the CDK inhibitor and doxorubicin are each administered in a therapeutically effective amount with respect to the individual components.

In another preferred embodiment of the invention, the CDK inhibitor and doxorubicin are each administered in a subtherapeutic amount with respect to the individual components.

Another aspect of the invention relates to the use of a CDK inhibitor and doxorubicin in the preparation of a medicament for treating a proliferative disorder.

Yet another aspect of the invention relates to the use of a CDK inhibitor in the preparation of a medicament for the treatment of a proliferative disorder, wherein said medicament is for use in combination therapy with doxorubicin.

A further aspect of the invention relates to the use of doxorubicin in the preparation of a medicament for the treatment of a proliferative disorder, wherein said medicament is for use in combination therapy with a CDK inhibitor.

As used herein, the term “combination therapy” refers to therapy in which the doxorubicin and CDK inhibitor are administered, if not simultaneously, then sequentially within a timeframe that they both are available to act therapeutically within the same time-frame.

As used herein the phrase “preparation of a medicament” includes the use of the components of the invention directly as the medicament in addition to their use in any stage of the preparation of such a medicament.

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the components of the present invention may induce apoptosis or maintain stasis within the desired cells as required.

Preferably, the proliferative disorder is a cancer or leukaemia, most preferably cancer.

In a particularly preferred embodiment, the invention relates to the use of the combination described herein in the treatment of a CDK dependent or sensitive disorder. CDK dependent disorders are associated with an above normal level of activity of one or more CDK enzymes. Such disorders preferably associated with an abnormal level of activity of CDK2 and/or CDK4. A CDK sensitive disorder is a disorder in which an aberration in the CDK level is not the primary cause, but is downstream of the primary metabolic aberration. In such scenarios, CDK2 and/or CDK4 can be said to be part of the sensitive metabolic pathway and CDK inhibitors may therefore be active in treating such disorders. Such disorders are preferably cancer or leukaemic disorders.

Preferably, the CDK inhibitor is an inhibitor of CDK2 and/or CDK4. More preferably the CDK inhibitor is selected from roscovitine, purvalanol A, purvalanol B, olomucine and other 2,6,9-trisubstituted purines as described in WO97/20842, WO98/05335 (CV Therapeutics), WO99/07705 (Regents of the University of California).

Even more preferably the CDK inhibitor is selected from roscovitine and purvalanol A.

In one particularly preferred embodiment, the CDK inhibitor is roscovitine. Roscovitine is the compound 2-[(1-ethyl-2-hydroxyethyl)amino]-6-benzylamine-9-isopropylpurine, also described as 2-(1-D,L-hydroxymethylpropylamino)-6-benzylamine-9-isopropylpurine. As used herein, the term “roscovitine” encompasses the resolved R and S enantiomers, mixtures thereof, and the racemate thereof.

Even more preferably, the combination has a synergistic effect, i.e., the combination is synergistic.

The effect of drug combinations is inherently unpredictable and there is often a propensity for one drug to partially or completely inhibit the effects of the other. The present invention is based on the surprising observation that administering doxorubicin and roscovitine in combination, either simultaneously, separately or sequentially, does not lead to any adverse interaction between the two agents. The unexpected absence of any such antagonistic interaction is critical for clinical applications.

Many anti-cancer agents are given in combination in order to optimise the treatment regime. For example, doxorubicin is frequently administered as part of cocktail or dosing regime of chemotherapeutic agents, see for example the database of chemotherapeutic combinations at http://www.indiacancer.org/prof/acd11.html. Doxorubicin has been proposed for use in sequential regime of docetaxal, doxorubicin and cyclophosphamide in the treatment of breast cancer (Journal of Clinical Oncology, Volume 19, No 14, pp 3367-3375, 2001). However, to date there has been no suggestion of administering doxorubicin in combination with roscovitine.

In a preferred embodiment, the combination of doxorubicin and roscovitine produces an enhanced effect as compared to either drug administered alone. The surprising nature of this observation is in contrast to that expected on the basis of the prior art.

As is well understood by those skilled in the field of cancer therapy, cancers may be classified not only by their location (breast, lung, kidney etc.) and nature (small cell, solid, soft etc.) but also biochemically on the basis of the genotype associated with a particular tumor. Considering a wide range of cancers, there are sub-populations within different cancers that exhibit p53 mutations. By way of example within a particular cancer, within breast cancer a subset based on genotypic characterisation would be those exhibiting a brca1 or brca2 mutation. As our understanding of the genotypic basis of different cancers increases, it is hoped that more specific treatments or treatment regimes can be developed.

In this regard, in one especially preferred embodiment of the invention, the proliferative disorder is an Rb deficient proliferative disorder. Experiments have unexpectedly shown that a combination of doxorubicin and roscovitine produced a maximal effect as compared to either drug administered alone upon administration to an Rb deficient cancer cell line. The surprising nature of this observation is in contrast to that expected, especially in view of the described activity of roscovitine in inhibiting Rb phosphorylation.

Thus, one preferred embodiment of the invention relates to a method of treating Rb deficient proliferative disorders comprising the administration of a therapeutically effective amount of CDK inhibitor and a therapeutically effective amount of doxorubicin.

Likewise, another preferred embodiment of the invention relates to the use of a CDK inhibitor in the manufacture of a medicament for use in the treatment of Rb deficient proliferative disorders comprising administration of a therapeutically effective amount of CDK inhibitor and a therapeutically effective amount of doxorubicin.

By way of definition, the term “Rb deficient” is used herein to refer to a genotype that is different from the wild-type Rb gene. Thus, the Rb genotype may be mutated or deleted, or the wild-type Rb gene may be present heterozygously. The reference taken for the wild-type Rb gene is that given in Lee et al (Lee W. H, Booksten, R, Hong, F D, Toung, L J, Shew J Y, Lee, E Y-H P, 1987; Human retinoblastoma susceptibility gene: cloning, identification and sequence. et al Science 235:1394-1399).

Restinoblastoma protein is a expressed by a gene originally identified in the context of an inherited eye tumor (Nevins J R, Human Molecular Genetics 2001, 10, 699-703). In its phosphorylated form it has been observed to be involved in the control of transcription factors such as E2F. Loss of Rb function has now been observed in a wide array of human cancers (Hunter T, Cell (1997) 88, 333-346, Sherr C J, (1996) Science 274, 1672-1677 and Weinberg R A (1995) Cell 81, 537-548). Thus, within the different types of cancer sub-populations have been observed exhibiting a mutation in the Rb gene, hereinafter referred to as Rb deficient cancers.

A recent report by Li et al (Cancer Research, Volume 61, No 6, pp 2579-82, 2001) showed that the osteosarcoma line SaOS-2, which is pRb-deficient, is significantly sensitised to the cytotoxic effects of doxorubicin when treated in combination with the CDK inhibitor flavopiridol. This effect was only seen in pRb-deficient cells, as SaOS-2 cells transfected with Rb did not show this enhanced sensitisation with the same drug combination. Cell cycle analysis revealed that flavopiridol decreased the doxorubicin-induced cell accumulation in the S-phase in Rb restored cells, but not in Rb-deficient cells. Increased p21waf-1 with concomitant decrease in cyclin-dependent kinase 2 kinase activity by flavopiridol was seen in SaOS-2 Rb deficient cells, but not in the Rb restored cells. A deficiency in PRb has also been reported for a liposarcoma cell line (Li et al. 1994 Br J Cancer 69, 1052-1058).

As discussed above, the use of a CDK inhibitor, preferably roscovitine, in combination with doxorubicin has been observed to be particularly effective in Rb deficient cancers. A comprehensive, but non-exhaustive list of cancers is given below. Patients exhibiting a particular cancer in an Rb deficient manner may be identified by a simple genotypic assay. Assays for decreased or increased Rb expression via immunohistochemistry are described in:
Xing E P, Guang-Yu, Y, Wang L-D, Shi, S T and Yang C S (1999) Loss of heterozygosity of the Rb gene correlates wit pRb protein expression and associates with p53 alteration in human esophageal cancer. Clinical Cancer Research 5:1231-1240
Geradts, J, Fong, K M, Zimmerman, P V, Maynard, R and Minna, J D (1999) Correlation of abnormal RB, p16^{ink4a}, and p53 expression with 3p loss of heterozygosity, other genetic abnormalities and clinical features in 103 primary non-small cell lung cancers

Thus, the present combination is particularly effective in the treatment of Rb deficient malignant and pre-neoplastic disorders. The invention is especially useful in relation to the treatment of Rb deficient adenocarcinomas such as: small cell lung cancer, and cancer of the kidney, uterus, prostrate, bladder, ovary, colon and breast. By way of example, malignancies which may be treatable according to the present invention include Rb deficient acute and chronic leukemias, lymphomas, myelomas, Rb deficient sarcomas such as fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, and the following cancers when present in the Rb deficient genotype: colon carcinoma, ovarian cancer, prostate cancer, pancreatic cancer, breast cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

Pharmaceutical Compositions

Although the components of the present invention (including their pharmaceutically acceptable salts, esters and pharmaceutically acceptable solvates) can be administered alone, for human therapy they will generally be administered in admixture with a pharmaceutical carrier, excipient or diluent.

A preferred embodiment of the invention therefore relates to a pharmaceutical composition comprising a CDK inhibitor and doxorubicin admixed with a pharmaceutically acceptable excipient, diluent or carrier.

Examples of such suitable excipients for the various different forms of pharmaceutical compositions described herein may be found in the “Handbook of Pharmaceutical Excipients, 2nd Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The agents of the present invention can be present as salts or esters, in particular pharmaceutically acceptable salts or esters.

Pharmaceutically acceptable salts of the agents of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The invention also includes where appropriate all enantiomers and tautomers of the agents. The man skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

Some of the agents of the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of agents, and mixtures thereof. The terms used in the claims encompass these forms, provided said forms retain the appropriate functional activity (though not necessarily to the same degree).

The present invention also includes all suitable isotopic variations of the agents or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agents of the present invention and pharmaceutically acceptable salts thereof can generally be prepared by conventional procedures using appropriate isotopic variations of suitable reagents.

Solvates

The present invention also includes solvate forms of the agents of the present invention. The terms used in the claims encompass these forms.

Polymorphs

The invention furthermore relates to agents of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes agents of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

Chemical Derivatives

The invention also relates to combinations which comprise derivatives of the agents. The term “derivative” as used herein includes chemical modification of an agent.

Illustrative of such chemical modifications would be replacement of hydrogen by a halo group, an alkyl group, an acyl group or an amino group.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredients can be incorporated into a cream consisting of an aqueous emulsion of polyethylene glycols or liquid paraffin. The active ingredients can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific agents employed, the metabolic stability and length of action of that agent, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, or from 2 to 20 mg/kg body weight. More preferably the agent may be administered at a dose of from 0.1 to 1 mg/kg body weight.

As described above, each active component, the CDK inhibitor and doxorubicin are preferably administered in therapeutically effective amounts, preferably in the form of pharmaceutically acceptable amounts. These amounts will be familiar to those skilled in the art. By way of guidance, doxorubicin is typically administered intravenously, or orally, most typically intravenously. Intravenous doses typically 60 mg/m² once every 21 days as a single agent or doses ranging from 20 to 30 mg/m² in combination therapies again once every 21 or 28 days. Further suitable dosing regimes may include 250 mg or 500 mg doxorubicin and are administered intravenously in accordance to a physicians direction at a total dosage depending on the weight of a patient e.g. orally at 15 mg/kg weekly, maximum dose 1 g/day, or intravenously 12 mg/kg over 4 hours, or 24-49 mg/kg over 24 hours daily for 5 days. Dosages and frequency of application are typically adapted to the general medical condition of the patient and to the severity of the adverse effects caused, in particular to those caused to the hematopoietic, hepatic and to the renal system.

Doxorubicin is available in several dosage forms; powder for reconstitution, in solution, as well as in lipid formulations encapsulated within liposomes. The skilled physician will be able to determine the most suitable dosage and dosage form for the particular patient and cancer type.

Roscovitine is typically administered orally or intravenously at a dosage of from about 0.05 to about 5g/day, preferably from about 0.5 to about 5 g/day or 1 to about 5 g/day, and even more preferably from about 1 to about 3 g/day. Alternatively, roscovitine is preferably administered at a dosage of about 0.4 to about 3 g/day. Roscovitine is preferably administered orally in tablets or capsules. The total daily dose of roscovitine can be administered as a single dose or divided into separate dosages administered two, three or four times a day.

The present invention is further described by way of example and with reference to the following figures wherein:

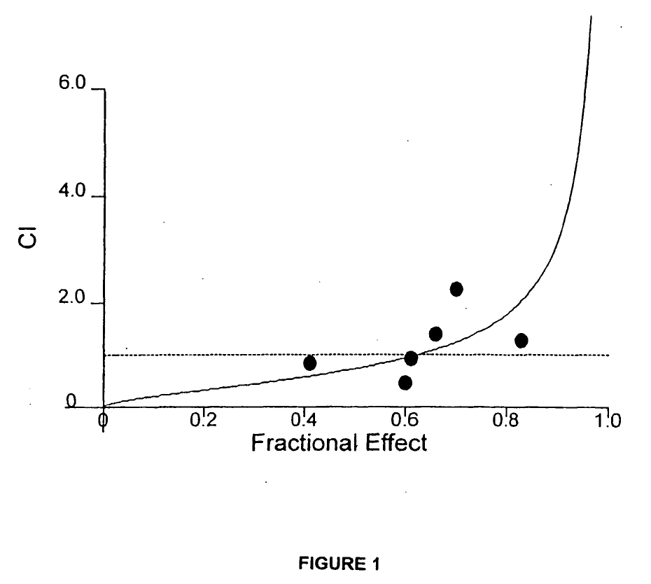

FIG. 1 shows the effects of combination treatment with doxorubicin and roscovitine usign median effect analysis: HTB88. Data obtained using the CalcuSyn program. Combination indices (CI)<1.0 are indicative of synergism.

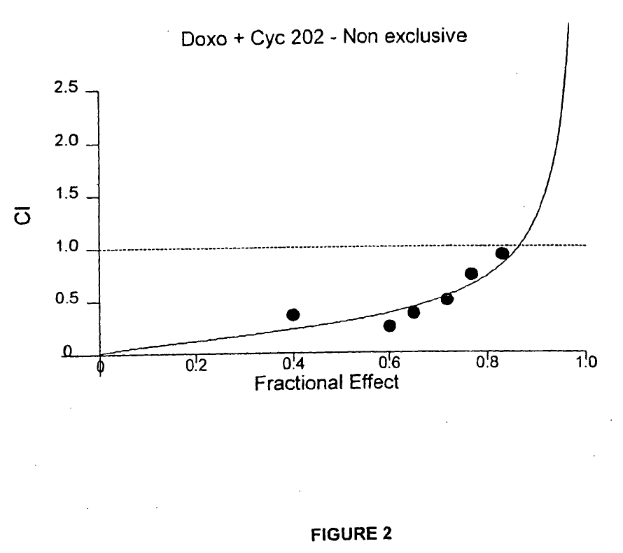

FIG. 2 shows the effects of combination treatment with doxorubicin and roscovitine usign median effect analysis: HTB114. Data obtained using the CalcuSyn program. Combination indices (CI)<1.0 are indicative of synergism.

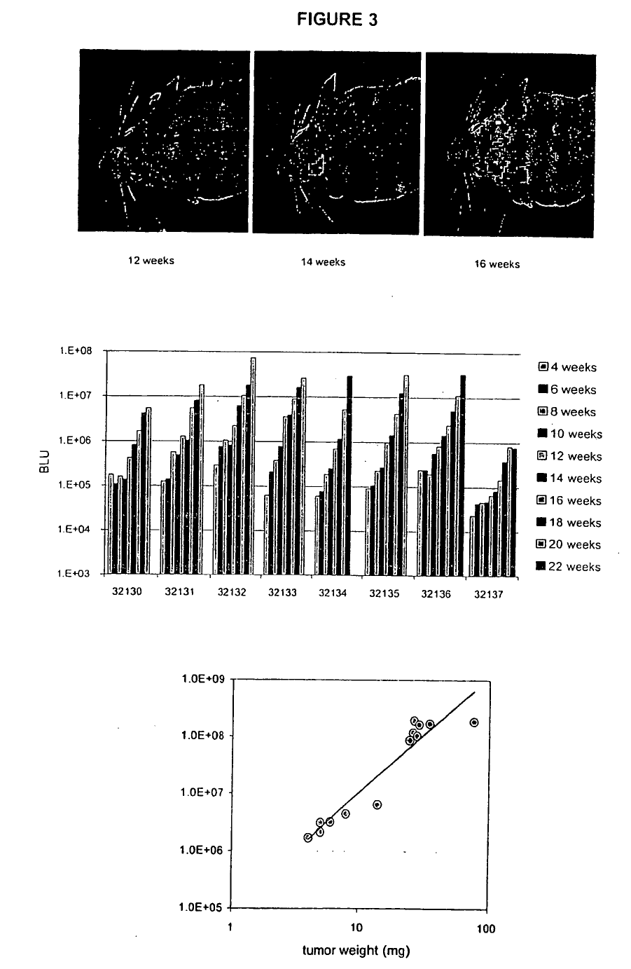

FIG. 3 shows tumour development in a POMC-Cre/POMC-Luc;Rb-flox/flox compound mutant mouse. Top: tumour development after 12, 14 and 16 weeks; Middle: bioluminescence (BLU) versus time, for eight different mice; Bottom: BLU versus tumour weight (mg).

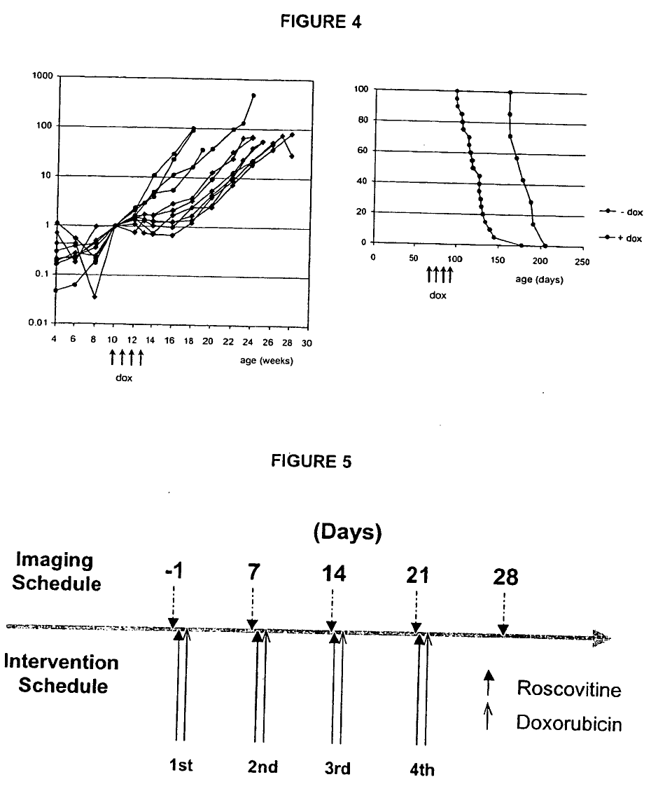

FIG. 4 shows the tumour response in POMC-Cre/POMC-Luc;Rb-flox/flox compound mutant mice to treatment with doxorubicin (4×5 mg/kg; intravenously).

FIG. 5 shows a drug administration schedule in which doxorubicin is administered once a weeks for four weeks (5 mg/kg; intravenously), and preceding each doxorubicin administration, roscovitine is administered orally twice daily for two days (500 mg/kg).

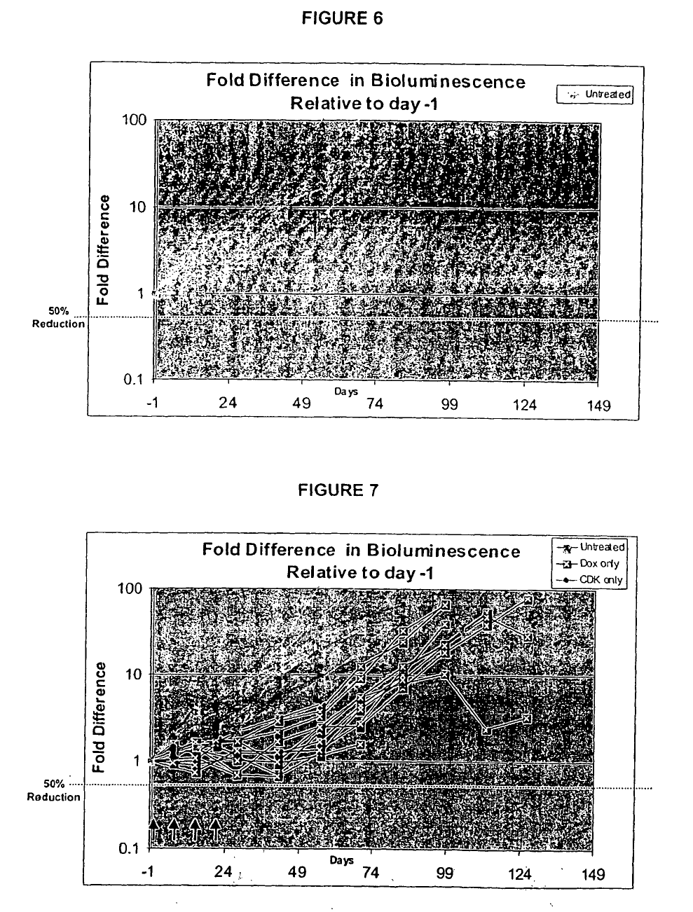

FIG. 6 shows the fold difference in bioluminscence relative to day 1 for untreated mice.

FIG. 7 shows the fold difference in bioluminscence relative to day 1 for untreated mice, and mice treated with either doxorubicin or roscivitine alone.

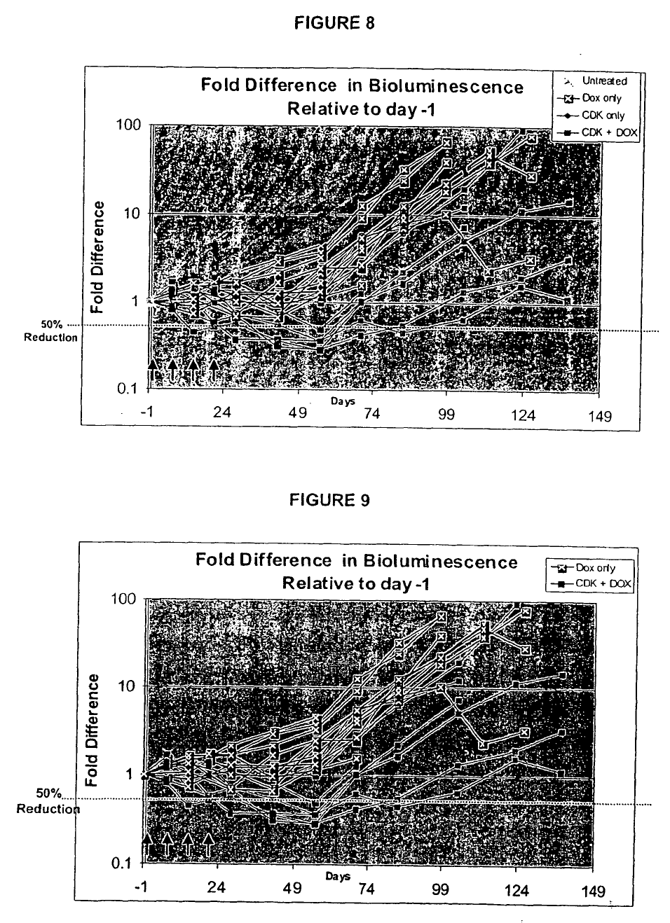

FIG. 8 shows the fold difference in bioluminscence relative to day 1 for untreated mice, mice treated with either doxorubicin or roscivitine alone, and mice treated with doxorubicin/roscovitine in combination.

FIG. 9 shows the fold difference in bioluminscence relative to day 1 for mice treated with doxorubicin alone, and mice treated with doxorubicin/roscovitine in combination.

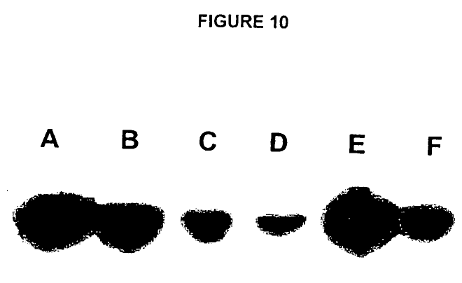

FIG. 10 shows Western immunoblotting analysis of Cyclin A; A: control (48 h); B: 10 μM roscovitine: C: 20 μM roscovitine; D: 5 nM doxorubicin: E: 10 nM doxorubicin; F: roscovitine/doxorubicin.

### EXAMPLES

Materials:

Doxorubicin and the tissue culture reagents are supplied by Sigma Aldrich. Roscovitine was supplied by Cyclacel Ltd. (Dundee UK). CalcuSyn program is from Cambridge Biosoft (2001/2002). HTB-88: SK-LMS-1 cell line was obtained from ATCC; human vulval primary leiomyosarcoma, p53 mutant (Patterson et al. (1994) Br J Cancer 69:1052-1058), pRb wild-type, p16 deleted. HTB-114: SK-UT-1 was obtained from ATCC; human uterine grade III leiomyosarcoma, P53 mutant (Patterson et al. (1994) supra.), pRb deleted, p16 wild-type.

Cytotox Method:

Cells at a density of 3×10⁴ cells /ml were seeded into 96-well plates in 200 μl aliquots. The medium was minimum essential medium with added non-essential amino acids and sodium pyruvate and 10% FCS (Life Technologies/Invitrogen). Cells were left for 24 h to equilibrate in a gassing humidifying incubator.

Stocks of drugs were made up in sterile water for doxorubicin and in DMSO for roscovitine and stored at −20° C. prior to use. Drugs were diluted in tissue culture medium (containing 10% FCS) to give the desired final concentrations. Drugs were added in 50 μl aliquots either singly or as a combination (i.e. roscovitine+doxorubicin). DOX was administered alone at stepwise dosages of from 10 nM to 320 nM. Roscovitine alone was administered alone at stepwise dosages of from 1.25 μM to 40 μM.

The drugs in combination were used at a fixed ratio to each other of DOX:roscovitine, 1:0.125, over the stepwise dilution series described above i.e starting at 10 nM DOX: 1.25 μM roscovitine to 320 nM DOX:40 μM roscovitine.

The cell viability was assessed following 72 h and 96 h for HTB114 and HTB88 respectively using the MTT method (according to Twentyman and Luscombe 1987, Br J Cancer 56: 279-285).

The combination index. (CI) was assessed according to the CacuSyn program based on the median effect analysis method.

Results:

The results are shown in FIGS. 1 and 2 which provide the combination index (CI) as calculated by the Calcusyn program. AS indicated a CI index <1 indicates a synergistic effect. A mild synergistic effect was observed on HTB-88 which correlated with a decrease in pRb phosphorylation. A marked synergistic effect was observed on HTB-114 (Rb deficient).

In Vivo Studies:

The effects of roscovitine and doxorubicin were investigated in a luminscent mouse model of pituitary tumours. A conditional mouse model for retinoblastoma-dependent sporadic cancer was generated (Vooijs et al; Cancer Res 2002 Mar 15; 62(6): 1862-7; 2002) which permits non-invasive monitoring of pituitary tumour development in live animals via in vivo bioluminescence imaging of luciferase expression. This mouse model permits the longitudinal monitoring of tumour onset, progression and response to therapeutic agents that specifically target the retinoblastoma pathway. By way of summary, a retinoblastoma conditional KO line was crossed with a POMC-Cre/POMC-Luciferase transgenic line to generate POMC-Cre/POMC-Luc;Rb-flox/flox compound mutant mice. These mice develop POMC (Rb deficient) tumours (Vooijs et al, ibid) and as illustrated in FIG. 3. Treatment with doxorubicin (4 x 5 mg/kg; intravenously) was shown to delay tumorigenesis. Doxorubicin is S-phase specific and temporarily halts tumour progression (see FIG. 4).

Once the baseline response to doxorubicin had been established, the model was used to investigate the effects of combination therapy using roscovitine and doxorubicin. In this study, doxorubicin was administered once a week for four weeks (5 mg/kg; intravenously). Preceding each doxorubicin adminstration, roscovitine was administered orally twice daily for two days (500 mg/kg) in accordance with the schedule illustrated in FIG. 5.

The fold difference in bioluminscence relative to day one is shown in FIG. 6 (untreated mice), FIG. 7 (untreated, doxorubicin and roscovitine (“CDK”) treated mice), FIG. 8 (untreated, doxorubicin, roscovitine, and doxorubicin/roscovitine treated mice), and FIG. 9 (doxorubicin, and doxorubicin/roscovitine treated mice). The Kruskal-Wallis Test (P<0.01) shows that the mean slopes from the four terminal treatment groups (FIG. 8) are not equal. Moreover, the Mann-Whitney Test (P<0.009) determines that there is a specific difference between the doxorubicin treated groups and the doxorubicin/roscovitine treated groups (FIG. 9).

In summary, the results indicate that doxorubicin temporarily halts tumour progression, and that pre-administration of roscovitine potentiates this doxorubicin effect, resulting in temporary tumour regression. This is the first time that the synergistic effect between doxorubicin and roscovitine has been demonstrated in vivo.

Western Immunoblotting:

Detection of protein levels was carried out using the NOVEX electrophoresis system using SDS-PAGE gels (10, 12 or 4-12%) and a MES or MOPS buffer. Whole cell lysates were prepared from appropriately treated cells harvested and prepared in TRIS buffer with SDS and protease inhibitors. An equal amount of protein was loaded in eachlane.

Results:

At 24 hours, a dose-dependent reduction in cyclin A was seen with roscovitine, with reduction in levels seen with the combination compared with doxorubicin alone in SK-UT-1 cells (see FIG. 10). Similar results were seen at 48 h.

Cell Cycle Analysis:

Flow cytometry for DNA cell cycle analysis: drug treated cells were harvested by centrifugation and washing in PBS, then fixed in 70% ethanol/PBS. Cells were stained with PI and treated with ribonuclease prior to flow cytometric analysis using FL3 with appropriate gating.

% of total (48 h treatment)

| Sample | Go | G1 | S | G2M |
|---|---|---|---|---|
| Control | 10 | 64 | 10 | 15 |
| Roscovitine | 23 | 36 | 16 | 19 |
| Doxo | 22 | 9 | 18 | 43 |
| Doxo + Roscovitine | 26 | 36 | 7 | 21 |

The results indicate that the addition of roscovitine to doxorubicin gives rise to a substantial reduction in the G2M peak seen with doxorubicin alone, pushing cells into GI. Cell cycle analysis showed a marked reduction in the doxorubicin-induced G2M block due to the addition of roscovitine in SK-UT-1 cells.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

1. A combination comprising a CDK inhibitor and doxorubicin.

2. A combination according to claim 1 wherein the CDK inhibitor is an inhibitor of CDK2 or CDK4.

3. A combination according to claim 1 or claim 2 wherein the CDK inhibitor is selected from rosovitine, purvalanol A, purvalanol B and olomoucine.

4. A combination according to any preceding claim wherein the CDK inhibitor is roscovitine.

5. A pharmaceutical composition comprising a combination according to any preceding claim and a pharmaceutically acceptable carrier, diluent or excipient.

6. Use of a combination according to any one of claims 1 to 4 in the preparation of a medicament for the treatment of a proliferative disorder.

7. Use according to claim 6 wherein the proliferative disorder is an Rb deficient proliferative disorder.

8. Use according to claim 6 wherein the proliferative disorder is a CDK-dependent or a CDK-sensitive disorder.

9. A pharmaceutical product comprising a CDK inhibitor and doxorubicin as a combined preparation for simultaneous, sequential or separate use in therapy.

10. A pharmaceutical product according to claim 9 wherein the CDK inhibitor is an inhibitor of CDK2 or CDK4.

11. A pharmaceutical product according to claim 9 or claim 10 wherein the CDK inhibitor is selected from rosovitine, purvalanol A, purvalanol B and olomoucine.

12. A pharmaceutical product according to any one of claims 9 to 11 wherein the CDK inhibitor is roscovitine.

13. A pharmaceutical product according to any one of claims 9 to 12 in the form of a pharmaceutical composition comprising a pharmaceutically acceptable carrier, diluent or excipient.

14. A pharmaceutical product according to any one of claims 9 to 13 for use in the treatment of a proliferative disorder.

15. A pharmaceutical product according to claim 14 wherein the proliferative disorder is an Rb deficient disorder.

16. A pharmaceutical product according to claim 14 wherein the proliferative disorder is cancer.

17. A method of treating a proliferative disorder, said method comprising administering to a subject, simultaneously, sequentially or separately, doxorubicin and a CDK inhibitor.

18. A method according to claim 17 which comprises administering said CDK inhibitor to a subject prior to sequentially or separately administering doxorubicin to said subject.

19. A method according to claim 17 which comprises administering doxorubicin to a subject prior to sequentially or separately administering a CDK inhibitor to said subject.

20. A method according to any one of claims 17 to 19 wherein the CDK inhibitor is an inhibitor of CDK2 or CDK4.

21. A method according to claim 20 wherein the CDK inhibitor is selected from rosovitine, purvalanol A, purvalanol B and olomoucine.

22. A method according to claim 21 wherein the CDK inhibitor is roscovitine.

23. A method according to any one of claims 17 to 22 wherein the CDK inhibitor and doxorubicin are each administered in a therapeutically effective amount with respect to the individual components.

24. A method according to any one of claims 17 to 22 wherein the CDK inhibitor and doxorubicin are each administered in a subtherapeutic amount with respect to the individual components.

25. A method according to any one of claims 17 to 24 wherein the proliferative disorder is an Rb deficient disorder.

26. A method according to any one of claims 17 to 24 wherein the proliferative disorder is cancer.

27. Use of a CDK inhibitor in the preparation of a medicament for the treatment of a proliferative disorder, wherein said treatment comprises administering to a subject simultaneously, sequentially or separately doxorubicin and a CDK inhibitor.

28. Use of a CDK inhibitor and doxorubicin in the preparation of a medicament for treating a proliferative disorder.

29. Use of a CDK inhibitor in the preparation of a medicament for the treatment of a proliferative disorder, wherein said medicament is for use in combination therapy with doxorubicin.

30. Use of doxorubicin in the preparation of a medicament for the treatment of a proliferative disorder, wherein said medicament is for use in combination therapy with a CDK inhibitor.

31. A combination comprising a CDK inhibitor and an anthracycline.
